(19) United States
(12) Patent Application Publication
(10) Pub. No.: US 2022/0401016 A1
(43) Pub. Date: Dec. 22, 2022

(54) Joint and Limb Monitoring System and Method Using Color Sensing

(71) Applicant: 3M INNOVATIVE PROPERTIES COMPANY, St. Paul, MN (US)

(72) Inventors: Jonathan B. Arthur, Hudson, WI (US); Thaine W. Fuller, Lakeland, MN (US); Nicholas G. Amell, Roseville, MN (US)

(21) Appl. No.: 17/755,873

(22) PCT Filed: Nov. 11, 2020
(86) PCT No.: PCT/IB2020/060630

§ 371 Date: May 10, 2022

Related U.S. Application Data
(60) Provisional application No. 62/937,090, filed on Nov. 18, 2019.

(51) Int. Cl. A61B 5/00; A61B 5/103; A61B 5/11; A61B 5/107

(52) U.S. Cl. CPC A61B 5/4528; A61B 5/1032; A61B 5/1114; A61B 5/1126; A61B 5/1121; A61B 5/0059; A61B 5/6833; A61B 5/7246; A61B 5/1071; A61B 2090/064

(57) ABSTRACT

Systems and methods for monitoring the status of a joint or limb using color sensing. First and second color sensors are provided to sense first and second color encoded surfaces at first and second locations of a joint or limb, respectively. The color sensing data are processed to obtain the respective motion information of the first and second locations.

### BACKGROUND

Medical and consumer grade braces or other wearable devices can provide compression and motion control for individuals with damaged or compromised joints and/or limbs. Braces are widely used for joints and limbs such as fingers, wrists, elbows, ankles, knees, necks, etc.

### SUMMARY

There is a desire to monitor or trace the status of a joint or limb. Add-ons can be provided to a wearable device (e.g., a brace, a bandage, etc.) worn by or attached to the joint or limb to monitor the status (e.g., force, motion, etc.) at a joint or a limb. The present disclosure provides systems and methods for monitoring the status of a joint or a limb using color sensing.

In one aspect, the present disclosure describes a method of monitoring a motion status of a joint or limb, the method comprising: providing a first color encoded surface at a first location of the joint or limb; providing a second color encoded surface at a second location of the joint or limb; providing a first color sensor facing the first color encoded surface; providing a second color sensor facing the second color encoded surface; obtaining, via the first and second color sensors, color sensing data from the first and second color encoded surfaces, respectively; and processing, via a processor, the color sensing data from the first and second color sensors to obtain the respective motion information of the first and second locations.

In another aspect, the present disclosure describes a system to monitor motion of a joint or limb, the system comprising: a first color encoded surface at a first location of the joint or limb; a second color encoded surface at a second location of the joint or limb; a first color sensor facing the first color encoded surface; a second color sensor facing the second color encoded surface. The first and second color sensors are configured to obtain color sensing data from the first and second color encoded surfaces, respectively. A computing device is configured to process the color sensing data from the first and second color sensors to obtain the respective motion information of the first and second location.

Various unexpected results and advantages are obtained in exemplary embodiments of the disclosure. One such advantage of exemplary embodiments of the present disclosure is that the monitoring systems and methods described herein allow for the precise measurement of joint or limb movement over time so that individuals and their medical providers (e.g., clinicians) can collect the accumulated objective data to enable treatment path evaluation and/or correction. To collect data from a joint or limb, conventional joint monitoring may have to be conducted manually in most clinical settings by having the patient move their limb or joint with or without the assistance of the clinician doing the evaluation.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

The disclosure may be more completely understood in consideration of the following detailed description of various embodiments of the disclosure in connection with the accompanying figures, in which:

FIG. 1 illustrates a schematic diagram of a system to detect material surfaces of a wearable object using color sensing, according to one embodiment.

FIG. 2 illustrates a flow diagram of a method for monitoring joints or limbs, according to one embodiment.

FIG. 3 illustrates a block diagram of a system to monitor joints or limbs, according to one embodiment.

FIG. 4 is an exploded perspective view of a monitoring device, according to one embodiment.

FIG. 5A illustrates a plan view of one major surface of a patch unit, according to one embodiment.

FIG. 5B illustrates a plan view of another major surface of the patch unit of FIG. 5A, according to one embodiment.

FIG. 5C illustrates a plan view of the major surface of the patch unit of FIG. 5B with deformation, according to one embodiment.

FIG. 6 illustrates a schematic diagram of a color sensor unit, according to one embodiment.

FIG. 7A illustrates a plan view of applying the patch unit of FIG. 5A onto a neck.

FIG. 7B illustrates a plan view of applying the color sensing device of FIG. 4 onto a neck.

FIG. 7C illustrates a schematic diagram of a sensing spot moving around a color encoded surface at various moments, according to one embodiment.

FIG. 8A illustrates a schematic diagram of a knee brace worn on a knee.

FIG. 8B illustrates a schematic diagram of a joint monitoring device applied to the knee brace of FIG. 8A.

FIG. 8C illustrates a schematic diagram of a color sensing device of FIG. 8B, according to one embodiment.

FIG. 9 illustrates a user interface on a mobile device for the color sensing device of FIG. 8B, according to one embodiment.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

Systems and methods for monitoring the status of a joint or a limb using color sensing are described. First and second color sensors are provided to sense first and second color encoded surfaces at first and second locations of a joint or limb, respectively. The color sensing data are processed to obtain the respective motion or status information of the first and second locations.

In some embodiments, a color encoded surface can include an area encoded with a gradient. For example, a color encoded surface can be shaded from black to gray with a gradient. The gradient might also have multiple colors with encoding from one region to another within a Red, Green, Blue measurement system or other color measurement system. Color encoding can also be facilitated through barcode patterns that create variability in color density across different areas of the encoded surface. The color encoding can also take the form of seemingly random color speckles, prints or patterns that have visible and non-visible color changes depending on the area of the encoded surface being measured. The colored patterns can be detected by visible or non-visible light.

FIG. 1 illustrates a schematic diagram of a system 100 to detect color information of a material surface 3 using color sensing, according to one embodiment. The system 100 includes a color sensor 10 configured to sense light reflected from the material surface 3. In some embodiments, the color sensor 10 can sense high precision color changes over time for various areas of the material surface 3. The color sensor 10 is disposed adjacent to the material surface 3 to determine various information of various targeted areas of the material surface 3 by detecting, e.g., changes in color within the targeted areas.

The material surface can be on the surface of a wearable object which is worn by a wearer. Exemplary wearable objects include a wearable brace, a compression sock, a bandage, a flexible wrap, a joint or limb support device, etc. The wearable object can include any suitable stretchable, compressible, or deformable materials such as, for example, a woven material, a nonwoven material (e.g., fibers), a foam material, etc., that is suitable to be worn by a wearer such as, for example, a person, a robot, an animal, or other wearers.

The material surface can be a color encoded surface providing color index for position information. A reference dataset such as, for example, a location matrix, can be predetermined by matching position (e.g., coordinates x and y in an x-y-z coordinate system) to a set of color values (e.g., RGB values). A color encoded surface can provide, for example, a color gradient where different positions (x, y) have different color values. A color encoded surface may include, for example, color fibers woven into a surface of a wearable object. A color encoded surface may include, for example, a topically colored area of a wearable object. A color encoded surface may include, for example, multiple woven layers of different color that is responsive to visible or non-visible light. The different layers may contribute to a color change upon a state change of the surface material, e.g., when a mechanical stress is applied to the material. A color encoded surface may include one or more surface coatings such as, for example, a coating of paint, pigment, dye, etc., on the surface of the material. Such a surface coating may contribute singularly or in combination with woven layers to the color change. A color encoded surface may include one or more back coatings visible to a color sensor described herein. It is to be understood that the various means to provide a color encoded surface can be combined and used for various color sensing applications.

The material surface may be stretchable, compressible, or deformable. While not wanting to be bound by theory, it is believed that a stretchable, compressible, or deformable material surface, such as a foam or an elastics surface, can change structurally (e.g., a change of porosity size, an exposure of underlying material, a damage to less-flexible materials, etc.) to induce a change of the spectrum and/or optical phase of the reflected light therefrom. For example, woven material surfaces may change in the distance between thread and elastic groupings depending on the direction of distortion, which can also change the spectrum and/or optical phase of the reflected light therefrom. In some cases, a targeted surface area of the wearable object may change its reflected wavelength (e.g., in the form of a material color change) during mechanical stress. Such a material color change can be readable by the system 100 of FIG. 1.

In some embodiments, the spectrum and/or optical phase change of light from the material surfaces of the wearable object can be derived from the displacement of the pigment in the material of the wearable object. The wearable object can include colored threads or films, and/or material modifications by other material processing techniques at various targeted areas of the wearable object. A color sensor can detect the corresponding spectrum or optical phase change when the wearable object is under a tension, a compression, a deformation, or a displacement.

In some embodiments, the spectrum and/or optical phase change of light from the material surfaces of the wearable object can be derived from the level of material wear of the wearable object. In some embodiments, at least a portion of the deformable material surfaces of the wearable object may change its color as the material wears. The material wear can include, for example, surface abrasion, deterioration of the material structure, etc., which can be detected by the measured color sensing data from the material surfaces.

In some embodiments, the material surfaces of the wearable object can include gradient layers of color. When the layers are changed (e.g., removed or damaged), the induced color change can be detected by the measured color sensing data from the material surfaces. In some embodiments, the material can be designed to express different levels of wear and types of damage through different color changes.

In some embodiments, the material surfaces of the wearable object can include a material having a critical wear warning label embedded in the material. The wear warning label might be a read layer that is not detectable by a color sensor unless being exposed through under certain level of wear.

The system 100 of FIG. 1 can digitally detect and quantify color changes on the surface of unmodified and modified material surfaces through visible or non-visible spectrum optical sensing. The measurement of the color changes for various surface areas of the wearable object allows for the quantification of distortion, pressure, damage, displacement, and motion that may or may not be visible to the human eye.

One or more color sensors 10 are functionally connected to a mobile device 20. The mobile device 20 can include a user interface (UI) to receive a user's instruction to obtain, via the color sensor 10, color sensing data of various target areas of the wearable object 3. The mobile device 20 can further include a computing element, e.g., a processor, to process the color sensing data from the color sensors 10 to obtain state information of various target areas of the wearable object 3. Exemplary state information may include tension, compression, deformation, displacement, level of material wear, etc. The user interface can then present the obtained state information to the user.

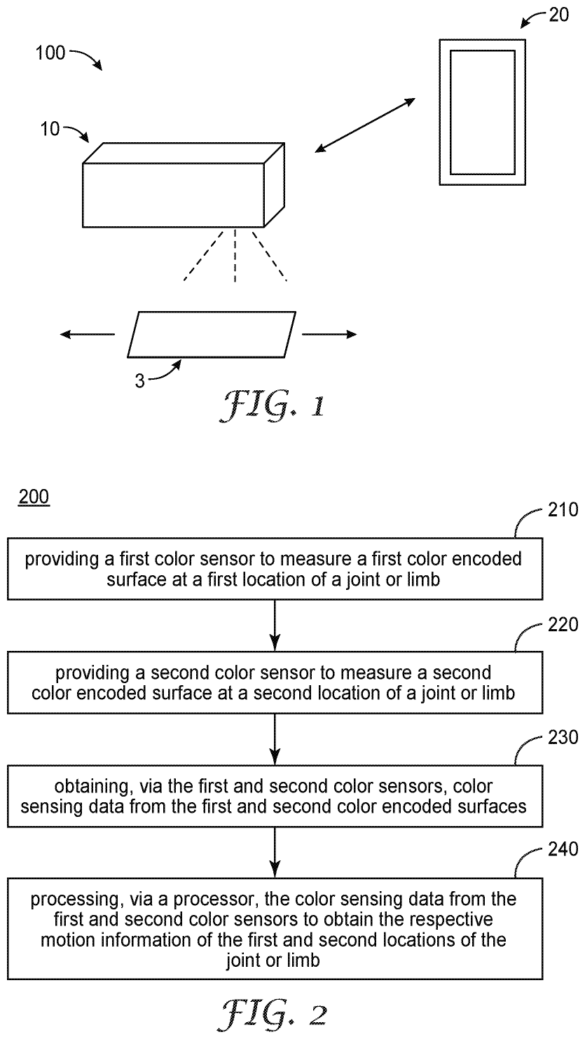

FIG. 2 illustrates a flow diagram of a method 200 to monitor the status (e.g., motion, force, etc.) of a joint or limb by color sensing, according to one embodiment. At 210, a first color sensor is provided to measure a first color encoded surface at a first location of the joint or limb. The first color encoded surface can be a material surface on a wearable object worn at the joint or a limb of a user. The wearable object can be, for example, a brace, a bandage, etc. In some embodiments, the first color sensor may be provided as one element of a color sensor pack including an optional light source to direct light to the first color encoded surface of the wearable object. The light source can be, for example, a white-colored LED positioned to illuminate at least a portion of the material surfaces. The first color sensor is positioned to sense the reflected light from the first color encoded surface. The method 200 then proceeds to 220.

At 220, a second color sensor is provided to measure a second color encoded surface at a second location of the joint or limb different from the first location. The second color encoded surface can be another material surface or another area of the material surface on the same wearable object worn at the joint or a limb. The first and second color sensors are respectively movable relative to the first and second color encoded surfaces when the joint or limb is in motion. In some embodiments, the second color sensor may be provided as one element of a color sensor pack including an optional light source to direct light to the second color encoded surface of the wearable object. The light source can be, for example, a white-colored LED positioned to illuminate at least a portion of the material surfaces. The second color sensor is positioned to sense the reflected light from the illuminated surface. In some embodiments, the first and second color sensors can be provided on a major side of the same sensor support. The light source can include a natural light source. The sensors can be positioned to allow for the reading of the White light to allow for changes in natural and unnatural light to be calibrated. Light from the light source can be channeled using gaps in the physical coverings, light conductive materials such as fiber optic glass or plastic, or surfaces used for reflection. The method 200 then proceeds to 230.

At 230, the first and second color sensors obtain color sensing data based on the sensed light reflected from the first and second color encoded surfaces, respectively. In some embodiments, the color sensing data obtained by a color sensor may include a digital return of color values such as, for example, red, green, blue, and white (RGBW) light sensing values, or red, green, blue (RGB) light sensing values. In some embodiments, the first and second color sensors can measure the color sensing data for the first and second locations at the same time. A time series of color sensing data can be obtained for the first and second locations, respectively. The method 200 then proceeds to 240.

At 240, a processor receives the color sensing data from the first and second color sensors and processes the color sensing data to obtain the respective motion or status information of the first and second locations. In some embodiments, the measured color sensing data can be analyzed and compared to a reference dataset to determine location information of the first and second color sensors with respect to the first and second color encoded surfaces. For example, an analytical module can compare measured color values to a reference dataset providing correspondences between the color sensing values (e.g., RGB values) and positions (e.g., x and y) for the first or second color encoded surfaces.

In some embodiments, a reference dataset can include a location matrix. The location matrix can include reference color values, e.g., red, green, blue, and white (RGBW) values, measured for various locations on the same material surface. The material surface can have, for example, a predetermined color distribution. The predetermined color distribution can provide correspondences between color values (RGBW values) and locations (e.g., X and Y coordinates in an X, Y coordinate system). It is to be understood that the reference dataset can be in any suitable forms other than a location matrix.

In some embodiments, the processor can calibrate the color sensors before use. For example, for a new material surface with unknow properties, color sensing data can be measured at known levels of tension/compression force to develop a location matrix providing between the color sensing values (e.g., RGB values) and positions (e.g., x and y) before using the new material surface as the first or second color encoded surfaces.

In some embodiments, the measured color sensing data and/or the determined position/motion information data can be stored in a database in any suitable data structure such as, for example, a table, an array, a matrix, etc. The data can be retrieved and analyzed to obtain useful information regarding the status of a joint or limb being monitored.

FIG. 3 illustrates a block diagram of a system 300 to monitor the status (e.g., motion, force, etc.) of the joint or limb using the method 200 of FIG. 2, according to one embodiment. The system 300 includes a first color sensor 310 configured to sense light reflected from the first color encoded surface at the first location of the joint or limb and obtain color sensing data based on the sensed light; and a second color sensor 320 configured to sense light reflected from the second color encoded surface at the second location of the joint or limb and obtain color sensing data based on the sensed light. One or more light sources can be integrated with the respective color sensors 310 and 320 to illuminate the respective color encoded surfaces at different locations of a joint or limb. In some embodiments, the color sensors 310 and 320 and their respective light sources can be integrated as a measurement unit 302, which can be the supported by the same sensor support. The measurement unit 302 may further include a controller 330 to allow control of the color sensors and the light sources. In some embodiments, the controller 330 may also provide analysis of the color sensing data from the color sensors. In some embodiments, the controller 330 may provide wire or wireless data communication with an external device such as, for example, a computing unit 304.

The measurement unit 302 is functionally connected to the computing unit 304. The computing unit 304 includes an analytic module (AM) 340 to process the color sensing data from the measurement unit 302 to determine state information of the joint or limb wearing a wearable object. The computing unit 304 further includes a user interface (UI) 350 to present information to a user and allow interaction with the user. The computing unit 304 can be integrated into a computer, a mobile device, or other computational devices.

The computing unit 304 can include a processor. The processor may include, for example, one or more general-purpose microprocessors, specially designed processors, application specific integrated circuits (ASIC), field programmable gate arrays (FPGA), a collection of discrete logic, and/or any type of processing device capable of executing the techniques described herein. In some embodiments, the processor (or any other processors described herein) may be described as a computing device. In some embodiments, the memory may be configured to store program instructions (e.g., software instructions) that are executed by the processor to carry out the processes or methods described herein. In other embodiments, the processes or methods described herein may be executed by specifically programmed circuitry of the processor. In some embodiments, the processor may thus be configured to execute the techniques for analyzing data related to a fluid network described herein. The processor (or any other processors described herein) may include one or more processors.

In some embodiments, the analytic module 340 can compare the obtained color sensing data to a reference dataset (e.g., a location matrix) to determine the location or motion information of one or more color encoded surfaces to be detected. The location matrix can include a matrix of color values for each (x, y) coordinate on the material surfaces. For each (x, y) coordinate, an array of color values may correspond to different deformation state of the corresponding position. The analytic module 340 can match the color sensing data to the closest color values in the matrix for the coordinates (x, y). The following Table 1 illustrates an exemplary location matrix for different positions, (x1, y1), (x2, y2), (x3, y3) on the material surface of a wearable object. For each position (e.g., Position 1, 2 or 3), there are arrows of measured color data (e.g., RGBW) values corresponding to different deformation state. Take Position 1 for example. The first row of RGBW values (3963,989,1630,999) corresponds to a state having low compression; the second row of RGBW values (3960,988,1630,980) corresponds to a state having proper compression; and the third row of RGBW values (3960,987,1631,975) corresponds to a state having too high compression.

TABLE 1

| | RGBW values |
|---|---|
| Position 1 | 3963, 989, 1630, 999 |
| (x1, y1) | 3960, 988, 1630, 980 |
| | 3960, 987, 1631, 975 |
| Position 2 | 3841, 1139, 1716, 902 |
| (x2, y2) | 3841, 1137, 1712, 850 |
| | 3840, 1135, 1710, 825 |
| Position 3 | 5606, 2950, 1682, 920 |
| (x3, y3) | 5606, 2925, 1678, 915 |
| | 5600, 2920, 1670, 902 |

In some embodiments, the analytic module 340 can first determine the location of the measured material surface (e.g., Position 1, 2 or 3 in Table 1) by matching the measured color sensing data to the closet range of reference color values of a location. For example, the analytic module 340 determines that a color reading (3961,987,1630,982) for a location best matches the color range of Position 1, then the analytic module 340 can determine the measured location to be Position 1. With the determined location (e.g., at Position 1), the analytic module 340 can match the measured color values to the nearest row of reference values for that position and to determine the corresponding deformation state. For example, the measured color values (3961,987,1630,982) for Position 1 best matches (3960,988,1630,980), which corresponds to a proper compression.

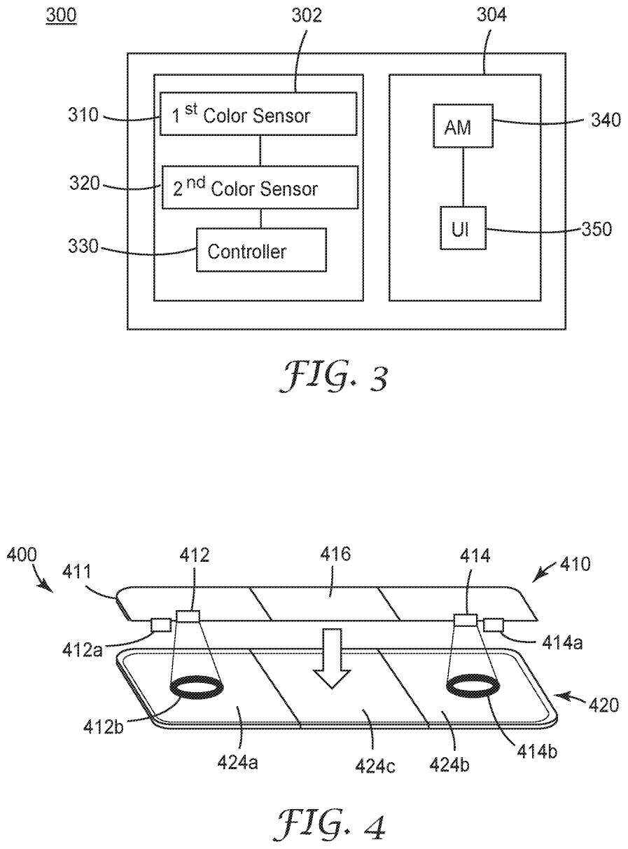

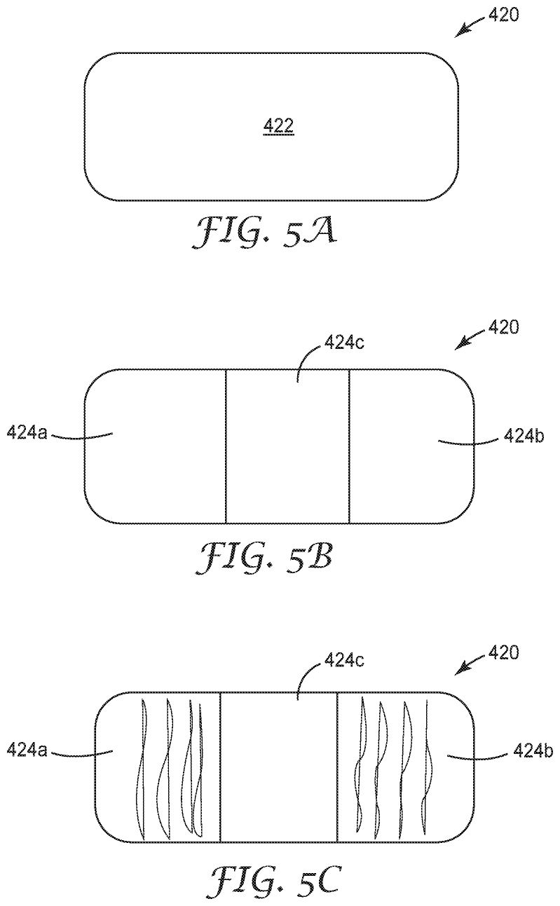

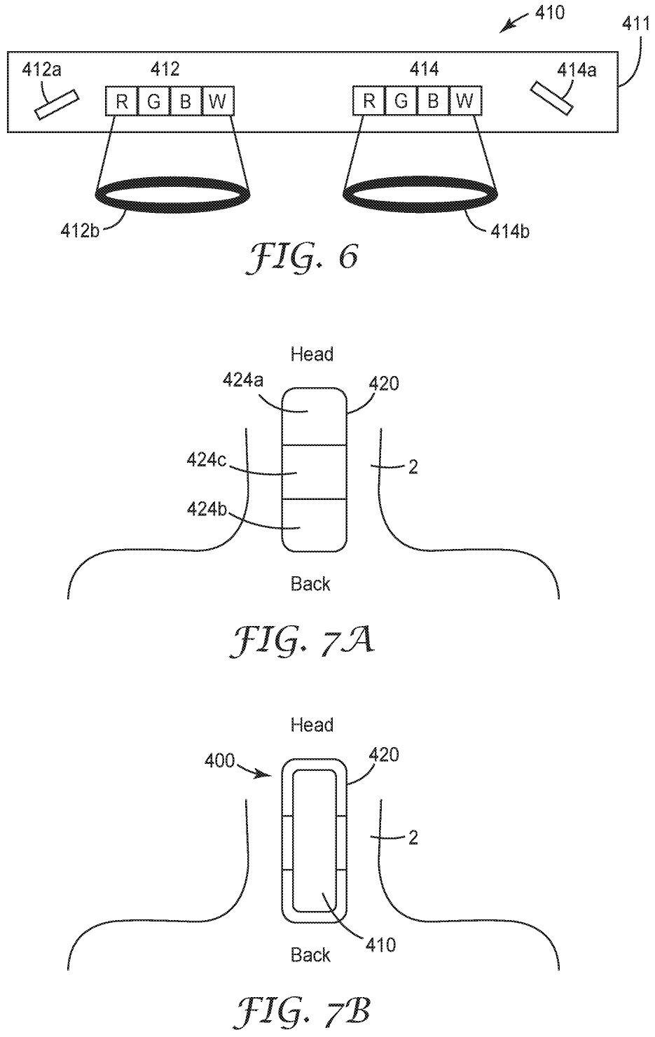

FIG. 4 illustrates a joint monitoring device 400, according to one embodiment. The joint monitoring device 400 includes a color sensor unit 410 and a patch unit 420. FIGS. 5A-B illustrate a bottom view and a top view of the patch unit 420, respectively. The patch unit 420 has an adhesive bottom surface 422 which is used for sticking to skin or to a material surface of a wearable object (e.g., a joint brace). The top surface 424 has a first color encoded surface 424a, a second color encoded surface 424b, and a connecting member 424c. FIG. 6 illustrates a schematic diagram of the sensor unit 410. The sensor unit 410 includes a sensor support 411 to support a first color sensor 412 and a second color sensor 414, and a connecting member 416. The sensor unit 410 and the patch unit 420 are connected via the respective connecting members 424c and 416 to form an anchor point.

In some embodiments, the connecting members 424c and 416 can form a separable connection. For example, the members 424c and 416 can include a layer of hook material and a layer of loop material, respectively, to form a hook and loop connector system. In some embodiments, at least one of the connecting members 424c and 416 can include adhesive to form adhesive bonding therebetween to secure the sensor unit 410 and the patch unit 420. It is to be understood that the connecting members 424c and 416 can have any suitable configurations to form the anchor point to secure the connection.

When the sensor unit 410 and the patch unit 420 are connected via the connecting members 424c and 416, the first color sensor 412 is positioned to face the first color encoded surface 424a of the patch unit 420; and the second color sensor 414 is positioned to face the second color encoded surface 424b of the patch unit 420. In the depicted embodiment of FIG. 4, light sources 412a and 414a are positioned adjacent to the respective color sensors 412 and 414 to illuminate the first and second color encoded surfaces 424a and 424b, respectively.

As shown in FIGS. 4 and 6A, the light from the first light source 412a reflects off the first sensing spot 412b on the first color encoded surface 424a and the light is received by the first sensor 412. The light from the second light source 414b reflects off the second sensing spot 414b on the second color encoded surface 424b and the light is received by the second sensor 414. The first and second color sensors 412 and 414 are configured to obtain color sensing data based on the sensed light reflected from the respective sensing spots 412b and 414b on the first and second color encoded surfaces 424a and 424b, respectively. In the depicted embodiment of FIG. 6A, the color sensing data obtained by the color sensors 412 and 414 include a digital return of red, green, blue, and white (RGBW) light sensing values. For example, an array of [110,32,94,45] with each value being red sensor value is 110, green sensor value is 32, blue sensor value is 94 and the white sensor value is 45. In some embodiments, the first and second color encoded surface 424a and 424b can have their respective primary colors and the first and second color sensors 412 and 414 can be designed to pick up the respective primary colors.

In some embodiments, the first and second color encoded surface 424a and 424b each can include colored threads or films, and/or material modifications by other material processing techniques. The color sensors can detect the corresponding spectrum or optical phase change when the color encoded surfaces are under a tension, a compression, a deformation, or a displacement. For example, when the patch unit 420 is deformed or stretched, additional threads may become visible that would not be visible with motion alone. In the depicted embodiment of FIG. 5C, the first and second color encoded surfaces 424a and 424b of the patch unit 420 are deformed lengthwise through stretching which exposes colored threads. For example, the original values of [110,93, 94,45] indicating the position may stay the same and the deformation can be measured.

While in the embodiment depicted in FIG. 4, the sensors 412 and 414 are disposed on the opposite sides of the anchor point, one or more anchor points formed by the connecting members 416 and 424c can be moved and modified in combination with sensor placement to minimize or maximize the measurement of specific movements depending on the desired purpose. One example may have one anchor point at the end of the sensor unit or support with one sensor to enable the measurement of movement. One or more anchor points can be disposed at either end of the device, placed in the middle of the device or in any combination to facilitate the measurement within a desired axis or set of axes.

The monitoring device can be modified to anchor the device and measure the axis of rotation for a given limb or body part. One example is the neck, where the monitoring device can be placed at the base of the neck above the shoulders with the sensor reading an encoded surface on the middle of the neck where the encoded surface is wrapped horizontally around the neck to measure the individual moving their head side to side.

In some embodiments, a monitoring device can be optimized in shape and form to allow for measurement of specific movement. These shapes might be in simple forms such as squares, rectangles or circles. Other form might include elongated ellipsis. The sensors, anchors and light sources might be incorporated in another system, such as a motorcycle helmet where the other system/device/article provides the function of a bracket with anchor points.

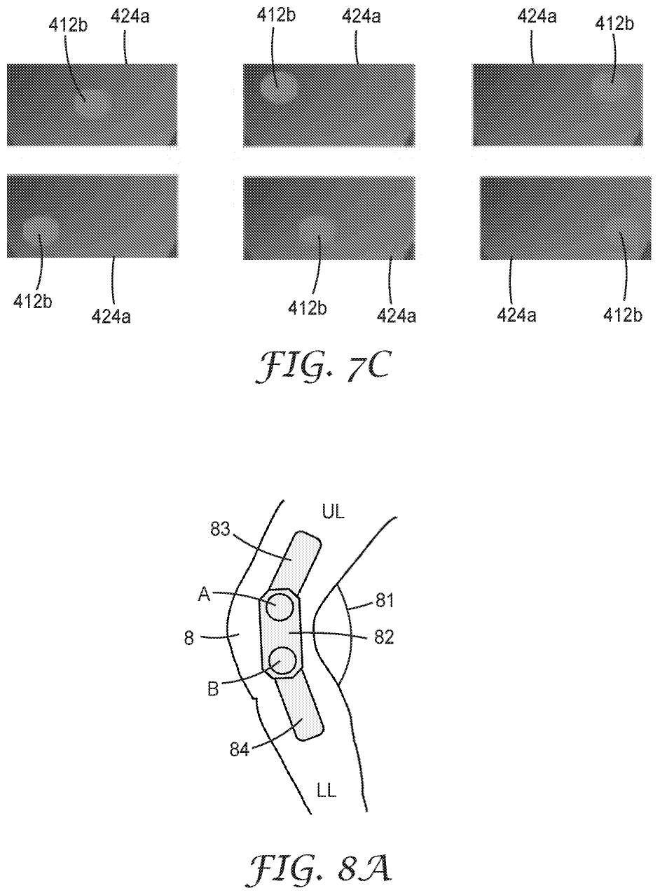

FIGS. 7A-B illustrate applying the monitoring device 400 to a neck 2. Individuals may have an issue with their necks with chronic pain. An area of interest can be identified for long-term monitoring and measurement in the middle of the individual's neck involving the spine. The patch unit 420 can be placed on the neck at the identified area for measurement. As shown in FIG. 7A, the patch unit 420 is placed on the neck with the area 2 of measurement in the center of the patch unit 420 in the direction of the expected problematic motion. The patch unit 420 has its adhesive bottom surface 422 sticking to the skin of the neck. As shown in FIG. 7B, the sensor unit 410 is disposed on the patch unit 420 by connecting the respective connecting members 424c and 416. See also, FIG. 4, where the first color sensor 414 is positioned to face the first color encoded surface 424a of the patch unit 420; and the second color sensor 414 is positioned to face the second color encoded surface 424b of the patch unit 420.

When the neck is under various motions (e.g., flexing, stretching, twisting, etc.), at least one of the first and second color encoded surfaces 424a and 424b of the patch unit 420 can move along with the neck. The sensor unit 410 is secured to the connection member 424c of the patch unit 420 and may not move along with the neck 2. In this manner, the first and second color sensors 412 and 414 of the sensor unit 410 can move around the respective first and second color encoded surfaces 424a-b. FIG. 7C illustrates the various motions of the sensing spot 412b of the first color sensor 412 around the first color encoded surface 424a when the neck flexes. The first and second color sensors 412 and 414 each can obtain a time series of color sensing data from different positions of the first and second color encoded surfaces 424a-b. The time series of color sensing data can be converted to a time series of position information which, in turn, can provide motion information of the neck. Such a conversion from color sensor data to position information may be conducted by comparing the color sensing data to a location matrix to determine the respective position information. For each determined position, deformation state at that position can be determined by comparing to reference color data values in a location matrix corresponding to different deformation state.

In measuring the use of a neck, a monitoring device including dual color sensors can measure various motions of a neck, including, for example, a simple bend of the neck (e.g., looking down) versus the jutting of the neck (e.g., moving the face forward relative to the correct position of the spine). The detected neck motions via the monitoring device can be used to determine posture over time or during any given time. This capability allows for the correction and understanding of the posture of an individual.

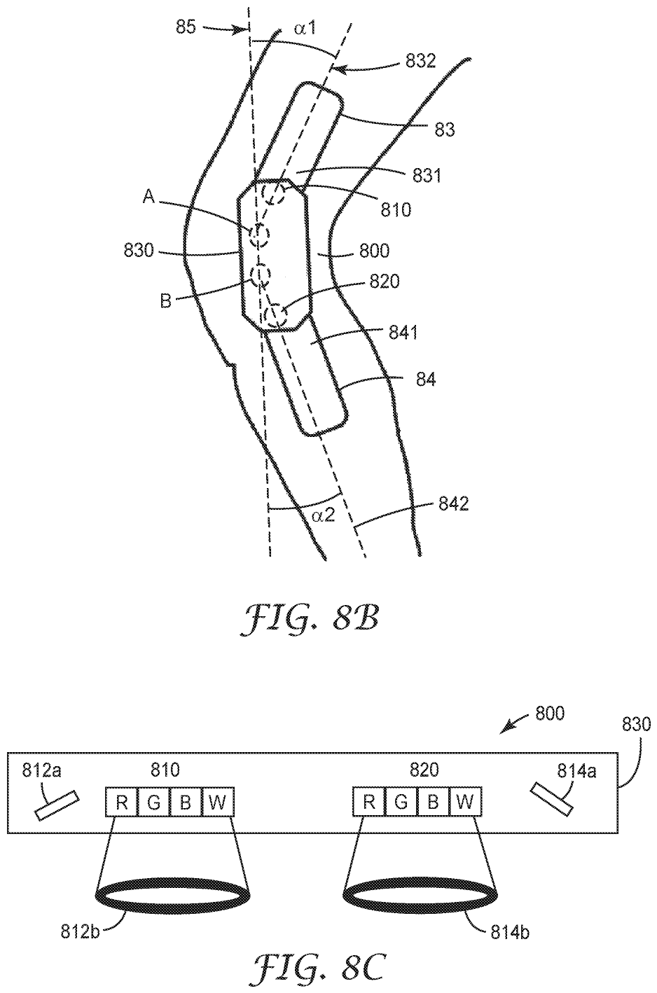

FIGS. 8A-D illustrate applying a joint monitoring device to a knee brace worn on a knee. As shown in FIG. 8A, a knee brace 8 includes a bracket 82 and two arms 83 and 84 pivotally connected to opposite ends of the bracket 82 at joints A and B to form a two-hinge construction. The upper arm 83 is attached to the upper leg and the lower arm 84 is attached to the lower leg. When the knee is under motion, the arms 83 and 84 may pivotally move relative to the bracket 82, and the angle 81 between the upper and lower arms 83 and 84 can change.

As shown in FIGS. 8B and 8C, a joint monitoring device 800 is applied to the knee brace 8 of FIG. 8A to monitor the motion of the knee. The joint monitoring device 800 includes a first color sensor 810 and a second color sensor 820 disposed on a bottom surface of an elongated sensor support 830. In the depicted embodiment of FIG. 8B, the color sensors are disposed adjacent to the opposite ends of the sensor support 830. A first color encoded surface 831 is provided to cover at least a portion of the upper arm 83 adjacent the joint A. A second color encoded surface 841 is provided to cover at least a portion of the lower arm 84 adjacent the joint B. The elongated sensor support 830 is attached to the bracket 82 such that the first color sensor 810 faces the first color encoded surface 831. Optional light sources 812a and 814a are provided to illuminate the first and second color encoded surfaces, respectively. The first color sensor 810 can receive color sensing data from a sensing spot 812b on the first color encoded surface 831. When the first color sensor 810 moves relative to the first color encoded surface 831, the sensing spot 812b moves around the first color encoded surface 831 and the measured color sensing data values change accordingly. The time series of color sensing data can be converted to the motion information of the upper leg portion connected to the knee. In a similar manner, the second color sensor 820 faces the second color encoded surface 841 covering at least a portion of the lower arm 84 at the joint B to detect the motion information of the lower leg portion connected to the knee. The second color sensor 820 can receive color sensing data from a sensing spot 814b on the second color encoded surface 841. When the second color sensor 820 moves relative to the second color encoded surface 841, the sensing spot 814b moves around the second color encoded surface 841 and the measured color sensing data values change accordingly. The time series of color sensing data can be converted to the motion information of the lower leg portion connected to the knee.

A connecting member such as, for example, the connecting member 416 in FIG. 4, can be provided to attach the joint monitoring device 800 to the bracket 82. The connecting member can be disposed on the bottom surface of the elongated sensor support 830 between the first and second color sensors 810 and 820. The connecting member may include a layer of hook or loop material to engage a layer of loop or hoop material attached to the surface of the bracket 82 to form a hook and loop connector system. The connecting member may include adhesive to form adhesive bonding to the surface of the bracket 82 to secure the elongated sensor support 830 thereon. It is to be understood that the connecting member can have any suitable configurations to form an anchor point to secure the connection.

When the knee is in motion, the first and second color sensors 810 and 820 of the can move around the respective first and second color encoded surfaces 831 and 841 adjacent to the joints A and B. The first and second color sensors 810 and 820 can take continuous color sensing data that provides the specific color values across the color encoded surface at any given time. The time series of color data values describe the relative position/motion of the sensors 810 and 820 in relation of the upper arm 83 and lower arm 84, respectively. The color sensing data can be received and processed by a computing device such as, for example, the computing unit 304 of FIG. 3.

Referring to the embodiment depicted in FIGS. 8A-C, the bracket 82 in this example is fixed relative to the face of the kneecap with the upper and lower legs moving in complex motion within the range of the two hinge bracket arms 83 and 84. Each encoded surface 831 and 841 has one color sensor 810 or 820 that reads position via the calculation made relative to the color encoded surface reading for each independent sensor.

The two hinge bracket arms 83 and 84 can accommodate the complex motion of the knee. They also allow for the distinction between different types of movement. For example, one type of movement is to bring the knee up vertically while standing, keeping the relative angle of the lower leg fixed. This is a distinctly different motion than a combined movement where the lower leg is moving in coordination with the upper leg to perform a task such as walking.

In some embodiments, an angle between first and second locations at a joint or limb can be determined based on the obtained motion information. In the depicted embodiment of FIG. 8B, the joint monitoring device 800 can capture the overall combined angle of the upper leg and lower leg at the knee. The upper leg angle a1 is measured between the bracket reference axis 85 and the first color encoded surface axis 832. The lower leg angle a2 is measured between the bracket reference axis 85 and the second color encoded surface axis 842. The bracket reference axis 85 can be a reference axis by connecting the joints A and B. The color encoded surface axis 831 or 841 can be a reference direction extending along the respective upper or lower leg.

The joint monitoring device 800 can monitor the angles at a joint or limb (e.g., angles a1 and/or a2 of FIG. 8B) for various types of joint or limb movement, which is more effective and convenient as compared current clinical practices. Clinical measurement is typically done in a clinician's office through manual manipulation of the limb (in this case the knee example), where the range of motion to bring the leg to a zero degree position, hyper extended position or bent position is evaluated from one extreme to another to understand the “range of motion”. The monitoring device described herein (e.g., the joint monitoring device 800) can provide a record of various angles at the joint or limb measured over time by taking multiple readings per minute or second. Such accumulated history data of motion for the joint or limb can be processed and present to assist a clinician and/or patient in understanding the use of the joint or limb.

The monitoring device or system described herein can assist a patient or clinician in understanding whether the range of motion of a joint or limb is adequate or not, e.g., whether the joint or limb is being used enough during a duration of time. The system can also assist in identifying deterioration of use (a reduction of range of motion) or if the individual is hyperextending the limb during movement.

The monitoring device or system described herein can also be used to measure the rate of motion of a joint or limb. The individual's joint or limb can be monitored for speed at which the joint or limb is being used at any given time. This can help with understanding whether the joint/limb is being overexerted or under exerted.

A monitoring device or system described herein such as the joint monitoring device 400 or 800 can be connected to a mobile device such as the mobile device 20 of FIG. 1 to monitor the status of a joint (e.g., a neck, a knee, etc.). the joint monitoring device can be functionally connected to the mobile device. The mobile device can include a computing unit such as the computing unit 304 of FIG. 3. The mobile device can run, via the computing unit, a mobile application to guide a user to control or interact with the joint monitoring device, and present status information regarding the joint or limb monitored by the joint monitoring device.

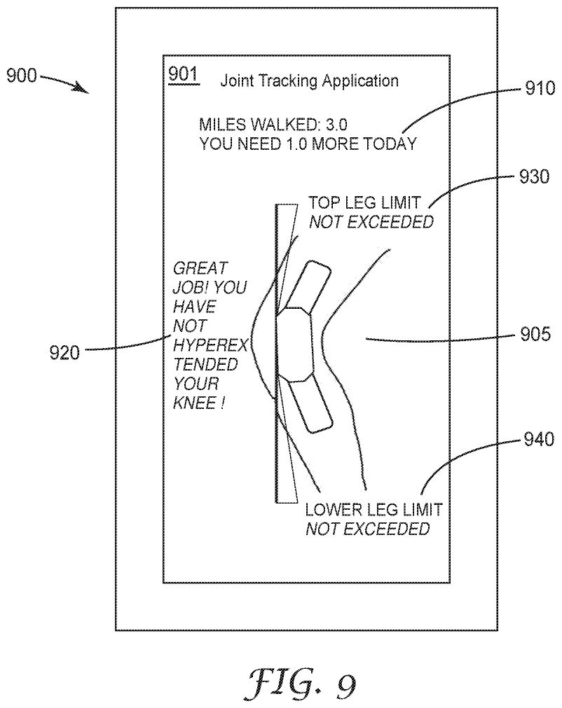

FIG. 9 illustrates a screenshot of a user interface provided by a joint monitoring application implemented by a computing device such as a computer or a mobile device. The user interface 900 includes a window 901 presenting information or data related to the knee 905 monitored by the joint monitoring device. The information may include, for example, status information 910 of the user, the status of the knee 920, the status of the top leg 930, the status of the lower leg 940, whether or not a user wearing the knee brace is over or under-extending their knee during walking or other exercise, etc. In the depicted embodiment, the user is being monitored for hyper-extension. The information shows that the user has not hyperextended the knee during the period and has followed the specified limits.

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

### Listing of Exemplary Embodiments

Exemplary embodiments are listed below. It is to be understood that any one of embodiments 1-10 and 11-18 can be combined.

Embodiment 1 is a method of monitoring a movement status of a joint or limb, the method comprising:

providing a first color encoded surface at a first location of the joint or limb;

providing a second color encoded surface at a second location of the joint or limb;

providing a first color sensor facing the first color encoded surface;

providing a second color sensor facing the second color encoded surface;

obtaining, via the first and second color sensors, color sensing data from the first and second color encoded surfaces, respectively; and

processing, via a processor, the color sensing data from the first and second color sensors to obtain the respective motion information of the first and second locations.

Embodiment 2 is the method of embodiment 1, further comprising providing a sensor support having an anchor member attached to the joint or limb.
Embodiment 3 is the method of embodiment 2, wherein the first and second color sensors are disposed on a major surface of the sensor support such that when the joint or limb moves, the first color sensor moves relative to the first color encoded surface, and the second color sensor moves relative to the second color encoded surface.
Embodiment 4 is the method of any one of embodiments 1-3, wherein obtaining the respective motion information comprises determining position information of the first and second color sensors with respect to the respective first and second color encoded surfaces based on the color sensing data.
Embodiment 5 is the method of embodiment 4, wherein determining the position information comprises comparing the color sensing data to a reference dataset to determine the respective position information.
Embodiment 6 is the method of embodiment 5, further comprising determining a compression or tension state of the respective first and second locations by comparing the color sensing data to the reference dataset.
Embodiment 7 is the method of any one of embodiments 1-6, further comprising determining an angle between the first and second locations at the joint or limb based on the obtained motion information.
Embodiment 8 is the method of embodiment 7, further comprising determining a range of motion and a rate of motion of the joint or limb based on the determined angle.
Embodiment 9 is the method of any one of embodiments 1-8, wherein the color sensing data from the color sensor comprises RGB values.
Embodiment 10 is the method of any one of embodiments 1-9, wherein the color sensor pack further comprises a light source configured to illuminate the material surfaces.
Embodiment 11 is a system to monitor motion of a joint or limb, the system comprising:

a first color encoded surface at a first location of the joint or limb;

a second color encoded surface at a second location of the joint or limb;

a first color sensor facing the first color encoded surface;

a second color sensor facing the second color encoded surface;

wherein the first and second color sensors are configured to obtain color sensing data from the first and second color encoded surfaces, respectively; and

wherein a computing device is configured to process the color sensing data from the first and second color sensors to obtain the respective motion information of the first and second locations.

Embodiment 12 is the system of embodiment 11, further comprising a sensor support having an anchor member attached to the joint or limb.
Embodiment 13 is the system of embodiment 12, wherein the first and second color sensors are disposed on a major surface of the sensor support such that when the joint or limb moves, the first color sensor moves relative to the first color encoded surface, and the second color sensor moves relative to the second color encoded surface.
Embodiment 14 is the system of embodiment 12 or 13, wherein the first and second color sensors are disposed on opposite sides of the anchor member.
Embodiment 15 is the system of any one of embodiments 11-14, further comprising an adhesive patch having an adhesive surface, wherein the first and second color encoded surfaces are disposed on a major surface of the adhesive patch opposite the adhesive surface.
Embodiment 16 is the system of embodiment 15, wherein the adhesive patch further comprises a connecting member to engage the anchor member of the sensor support.
Embodiment 17 is the system of any one of embodiments 11-16, wherein the first and second color encoded surfaces each include a color gradient.
Embodiment 18 is the system of any one of embodiments 11-17, further comprising one or more light sources configured to illuminate the first and second color encoded surfaces.
Reference throughout this specification to “one embodiment,” “certain embodiments,” “one or more embodiments,” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus, the appearances of the phrases such as “in one or more embodiments,” “in certain embodiments,” “in one embodiment,” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments. While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. Furthermore, various exemplary embodiments have been described. These and other embodiments are within the scope of the following claims.

1. A method of monitoring a joint or limb, the method comprising:
providing a first color encoded surface at a first location of the joint or limb;
providing a second color encoded surface at a second location of the joint or limb;
positioning a first color sensor facing the first color encoded surface;
positioning a second color sensor facing the second color encoded surface;
obtaining, via the first and second color sensors, color sensing data from the first and second color encoded surfaces, respectively; and
processing, via a processor, the color sensing data from the first and second color sensors to obtain the respective motion information of the first and second locations.

2. The method of claim 1, further comprising providing a sensor support having an anchor member attached to the joint or limb.

3. The method of claim 2, wherein the first and second color sensors are disposed on a major surface of the sensor support such that when the joint or limb moves, the first color sensor moves relative to the first color encoded surface, and the second color sensor moves relative to the second color encoded surface.

4. The method of claim 1, wherein obtaining the respective motion information comprises determining position information of the first and second color sensors with respect to the respective first and second color encoded surfaces based on the color sensing data.

5. The method of claim 4, wherein determining the position information comprises comparing the color sensing data to a reference dataset to determine the respective position information.

6. The method of claim 5, further comprising determining a compression or tension state of the respective first and second locations by comparing the color sensing data to the reference dataset.

7. The method of claim 1, further comprising determining an angle between the first and second locations at the joint or limb based on the obtained motion information.

8. The method of claim 7, further comprising determining a range of motion and a rate of motion of the joint or limb based on the determined angle.

9. The method of claim 1, wherein the color sensing data from the color sensor comprises RGB values.

10. The method of claim 1, wherein the color sensor pack further comprises a light source configured to illuminate the material surfaces.

11. A system to monitor motion of a joint or limb, the system comprising:
a first color encoded surface at a first location of the joint or limb;
a second color encoded surface at a second location of the joint or limb;
a first color sensor facing the first color encoded surface; and
a second color sensor facing the second color encoded surface,
wherein the first and second color sensors are configured to obtain color sensing data from the first and second color encoded surfaces, respectively, and
wherein a computing device is configured to process the color sensing data from the first and second color sensors to obtain the respective motion information of the first and second locations.

12. The system of claim 11, further comprising a sensor support having an anchor member attached to the joint or limb.

13. The system of claim 12, wherein the first and second color sensors are disposed on a major surface of the sensor support such that when the joint or limb moves, the first color sensor moves relative to the first color encoded surface, and the second color sensor moves relative to the second color encoded surface.

14. The system of claim 12, wherein the first and second color sensors are disposed on opposite sides of the anchor member.

15. The system of claim 11, further comprising an adhesive patch having an adhesive surface, wherein the first and second color encoded surfaces are disposed on a major surface of the adhesive patch opposite the adhesive surface.

16. The system of claim 15, wherein the adhesive patch further comprises a connecting member to engage the anchor member of the sensor support.

17. The system of claim 11, wherein the first and second color encoded surfaces each include a color gradient.

18. The system of claim 11, further comprising one or more light sources configured to illuminate the first and second color encoded surfaces.
